(19) United States
(12) Patent Application Publication
(10) Pub. No.: US 2016/0160237 A1
(43) Pub. Date: Jun. 9, 2016

(54) Mitochondrial Collection and Concentration, and Uses Thereof

(71) Applicant: OvaScience, Inc., Waltham, MA (US)

(72) Inventors: Colleen M. BURGESS, Scituate, MA (US); Scott CHAPPEL, Milton, MA (US); Arthur O. TZIANABOS, Reading, MA (US)

(21) Appl. No.: 14/959,130

(22) Filed: Dec. 4, 2015

Related U.S. Application Data
(60) Provisional application No. 62/089,118, filed on Dec. 8, 2014.

(51) Int. Cl. C12N 15/877; B01D 21/26; B01D 17/02

(52) U.S. Cl. CPC C12N 15/8776; B01D 17/0217; B01D 21/262

(57) ABSTRACT

Preparations and methods of producing a concentrated mitochondrial preparation using inverted phase centrifugation are disclosed. The methods include centrifuging a closed-bottom tube containing a heavy phase including a mitochondrial suspension in the portion of the tube proximal to the axis of rotation and a light phase in the portion of the tube distal to the axis of rotation. The centrifugation causes the suspension to form a concentrated mitochondrial layer at the interface between the heavy layer and the light layer, and the fluid at this interface is collected to produce the concentrated mitochondrial preparation. The concentrated mitochondrial preparation can be used for various applications, including in vitro fertilization applications.

### CROSS REFERENCE TO RELATED APPLICATIONS

This application claims priority to U.S. Provisional Patent Application No. 62/089,118, filed Dec. 8, 2014, the contents of which are incorporated by reference in its entirety.

### BACKGROUND OF THE INVENTION

1. Field of the Invention

The invention relates to the collection of mitochondria from mammalian cells, the concentration of the mitochondria, and uses of the concentrated mitochondrial preparations.

2. Description of the Related Art

Mitochondria are membrane-bound organelles found in most eukaryotic cells.

Mitochondria range from 0.5 μm to 1.0 μm in diameter. Mitochondria provide energy in the form of adenosine triphosphate (ATP) for most intracellular events. Mitochondria also have important functions in ion homeostasis, programmed cell death, and adaptive thermogenesis. Mitochondrial dysfunction has been implicated in a number of pathophysiological processes such as aging, neurodegenerative diseases, diabetes, obesity, and infertility. Aging is associated with a decrease in mitochondrial function, especially in non-replicating cells such as the mature oocyte, and decreased mitochondrial function is considered one cause for declining fertility in older women.

Although most of a cell's DNA is contained in the cell nucleus, mitochondria have their own independent genome. In addition, unlike nuclear chromosomal DNA, there are multiple copies of the mitochondrial genome in each mitochondrion, with an average of five mtDNA genomes per organelle in somatic cells. The mitochondrial DNA (mtDNA) is a maternally inherited double-stranded circular genome that encodes 37 genes, including 13 for polypeptides involved in respiratory and oxidative phosphorylation. The human metaphase oocyte is estimated to have 100,000-200,000 mitochondria. Tzeng et al. (2004), Fertil. Steril. 82(S2), S53. Unlike eukaryotic nuclear DNA, mtDNA lacks noncoding introns and histones, and is more susceptible to damage by high concentrations of reactive oxygen species (ROS) and free radicals in the matrix of mitochondria. As a result of long-lasting exposure to ROS, oocyte mitochondria accumulate mtDNA mutations and deletions. With aging, exposure of the mitochondrial genome to ROS increases, which compromises mitochondrial function and causes a decrease in energy availability in oocytes.

To improve the reproductive outcome with aging oocytes, attempts have been made to inject a mitochondrial concentrate into oocytes to increase mitochondrial function, and thereby increase fertilization rates and/or enhance embryonic development. For example, mitochondria from young mouse oocytes have been transferred into mature older oocytes. Perez et al. (2000), Nature, 403:500-1. However, mitochondrial transfer from non-autologous cells resulted in mitochondrial heteroplasmy (i.e., different mitochondrial genomes in the same cell) and an abnormal mouse phenotype. Attempts in humans to perform ooplasmic transfers (i.e., transfers of oocyte cytoplasm, including mitochondria) from young donor ova into low-quality oocytes from an older patient were discontinued because this approach caused mitochondrial heteroplasmy in the offspring. Brenner et al. (2001), Fertil. Steril. 74:573-8; Barrit et al. (2001), Hum. Reprod. 16:513-16. Mitochondrial transfer into oocytes from autologous cells would avoid the problem of heteroplasmy of donor and recipient mtDNA.

Prior art methods of mitochondrial collection have produced mitochondrial preparations with lower concentrations of mitochondria and/or less purified preparations of mitochondria and/or a larger proportion of damaged mitochondria with, for example, disruptions to the integrity of the outer mitochondrial membrane and/or partial loss of the respiratory and/or energy-production functions of mitochondria. Therefore, there remains a need in the art for better methods for preparing concentrated mitochondrial preparations with a high proportion of functional mitochondria.

### SUMMARY OF THE INVENTION

The present invention depends, in part, upon the discovery of methods of collecting and concentrating mitochondria without the use of dyes, chromophores, fluorophores or other physical, chemical or metabolic markers. In particular, the method utilizes inverted phase centrifugation, in which a mitochondria-containing heavy phase remains proximal and a light phase remains distal to the axis of rotation, and permits collection of a concentrated mitochondrial preparation at the phase boundary. The mitochondrial preparation are characterized by a degree of concentration and high mitochondrial function that is superior to the prior art.

The concentrated mitochondrial preparations can be used for various applications, including use for mitochondrial transfers into oocytes (“Autologous Germline Mitochondrial Energy Transfer”^{SM} or AUGMENT^{SM}, OvaScience, Inc., Waltham, Mass.) for in vitro fertilization. The AUGMENT^{SM} process includes transferring a composition comprising mitochondria from mammalian female germline stem cells or oogonial stem cells (OSC), or mitochondria obtained from a progeny of an OSC, into an autologous oocyte, thereby preparing the oocyte for IVF or artificial insemination. The AUGMENT^{SM} process is described in WO 2012/142500, entitled “Compositions and Methods for Autologous Germline Mitochondrial Energy Transfer,” filed Apr. 13, 2012, which is incorporated in its entirety herein.

In one aspect, the present disclosure relates to methods for producing a concentrated mitochondrial preparation including (a) centrifuging a closed-bottom tube containing (i) a heavy phase comprising a mitochondrial suspension in the portion of the tube proximal to the axis of rotation; (ii) a light phase in the portion of the tube distal to the axis of rotation; and (iii) a phase interface between the heavy phase and the light phase. In these methods, the closed-bottom tube is centrifuged at a speed and for a time sufficient to cause mitochondria in the mitochondrial suspension to form a concentrated mitochondrial layer in the heavy phase at the interface between the light phase and the heavy phase, and without causing inversion of the light and heavy phases. The methods further include (b) collecting a volume of fluid comprising at least a portion of the concentrated mitochondrial layer to produce the concentrated mitochondrial preparation.

In another aspect, the present disclosure relates to methods for producing a concentrated mitochondrial preparation including (a) drawing a volume of a first fluid including a mitochondrial suspension into an open-bottom tube. The methods further include (b) drawing a volume of a second fluid into the tube, wherein the second fluid is less dense than the first fluid, thereby forming a heavy phase comprising the first fluid and mitochondrial suspension in one portion of the tube, a light phase comprising the second fluid in another portion of the tube, and a phase interface between the heavy phase and the light phase. The methods further include (c) sealing the end of the tube proximal to the light phase to form a closed-bottom tube; and (d) placing the closed-bottom tube in the centrifuge with the sealed end distal to the axis of rotation. The methods further include (e) centrifuging the closed-bottom tube at a speed and for a time sufficient to cause mitochondria in the mitochondrial suspension to form a concentrated mitochondrial layer in the heavy phase at the interface between the light phase and the heavy phase, and without causing inversion of the light and heavy phases. The methods further include (f) collecting a volume of fluid comprising at least a portion of the concentrated mitochondrial layer to produce the concentrated mitochondrial preparation.

In some embodiments, in the disclosed methods, the first fluid is drawn into the tube before the second fluid is drawn into the tube.

In some embodiments, in the disclosed methods, the heavy phase is an aqueous phase.

In some embodiments, in the disclosed methods, the heavy phase is obtained by chemical, mechanical or sonic lysis of cells, followed by centrifugation.

In some embodiments, in the disclosed methods, the light phase is an organic phase.

In some embodiments, in the disclosed methods, the light phase comprises a biocompatible oil.

In some embodiments, in the disclosed methods, the biocompatible oil light is selected from the group consisting of a mineral oil, a paraffin oil, a light oil, a cell culture oil and an ICSI oil.

In some embodiments, in the disclosed methods, the concentrated mitochondrial preparation has a concentration of at least 1 mitochondria per picoliter.

In some embodiments, in the disclosed methods, the concentrated mitochondrial preparation has a mitochondrial concentration of between 1 and 10,000 mitochondria per picoliter; between 50 and 10,000 mitochondria per picoliter; between 100 and 10,000 mitochondria per picoliter; between 150 and 10,000 mitochondria per picoliter; between 200 and 10,000 mitochondria per picoliter; between 250 and 10,000 mitochondria per picoliter; between 300 and 100,000 mitochondria per picoliter; between 400 and 10,000 mitochondria per picoliter; between 450 and 10,000 mitochondria per picoliter; and between 500 and 10,000 mitochondria per picoliter.

In some embodiments, in the disclosed methods, the tube has an inner diameter at the interface between the heavy phase and the light phase of less than 1 μm.

In some embodiments, in the disclosed methods, the tube is centrifuged at a rate of less than 10,000 G.

In some embodiments, in the disclosed methods, the tube is centrifuged at a rate between 7,500 and 12,500×G.

In some embodiments, in the disclosed methods, after drawing the volume of the second fluid into the open-bottom tube, the portion of the tube proximal to the light phase is heat sealed across the portion containing the light phase to form a closed-bottom tube.

In some embodiments, in the disclosed methods, the tube comprises a material selected from the group consisting of polyethylenes (PE), high-density polyethylenes (HDPE), low-density polyethylenes (LDPE), polyethylene terephthalates (PET), polypropylenes (PP), polyvinyl chlorides (PVC), polyvinylidene chlorides (PVDC), polystyrenes (PS), high impact polystyrene (HIPS), acrylonitrile butadiene styrenes, polyamides, polycarbonates, polyurethanes and nylons.

In some embodiments, in the disclosed methods, at least 90%, 91%, 92%, 93%, 94% 95%, 96%, 97%, 98%, 99%, or 99.5%% of the mitochondria in the concentrated mitochondrial preparation are functional.

In another aspect, the present disclosure relates to concentrated mitochondrial preparations comprising a solution including at least one mitochondria per picoliter.

In some embodiments, in the disclosed preparations, the solution includes between 1 and 10,000 mitochondria per picoliter. In some embodiments, in the disclosed preparations, the solution includes between 1 and 10,000 mitochondria per picoliter; between 50 and 10,000 mitochondria per picoliter; between 100 and 10,000 mitochondria per picoliter; between 150 and 10,000 mitochondria per picoliter; between 200 and 10,000 mitochondria per picoliter; between 250 and 10,000 mitochondria per picoliter; between 300 and 100,000 mitochondria per picoliter; between 400 and 10,000 mitochondria per picoliter; between 450 and 10,000 mitochondria per picoliter; and between 500 and 10,000 mitochondria per picoliter.

In some embodiments, in the disclosed preparations, the preparation further comprises a volume of a biocompatible oil.

In some embodiments, in the disclosed preparations, at least 90%, 91%, 92%, 93%, 94% 95%, 96%, 97%, 98%, 99%, or 99.5% of the mitochondria are functional.

These and other aspects and embodiments of the disclosure are illustrated and described below. Other systems, processes, and features will become apparent to one with skill in the art upon examination of the following drawings and detailed description. It is intended that all such additional systems, processes, and features be included within this description, be within the scope of the present invention, and be protected by the accompanying claims.

### BRIEF DESCRIPTION OF THE DRAWINGS

The following drawings are illustrative of embodiments of the invention and are not meant to limit the scope of the invention as encompassed by the claims.

FIG. 1 is a schematic diagram that shows some embodiments of methods for producing a concentrated mitochondrial preparation.

FIG. 2 is a schematic diagram that shows other embodiments of methods for producing a concentrated mitochondrial preparation.

### DETAILED DESCRIPTION

The present disclosure relates to the collection of mitochondria from mammalian cells, the concentration of the mitochondria, and uses of the concentrated mitochondrial preparations.

### DEFINITIONS

All scientific and technical terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the subject matter which is the invention, the following definitions are provided for certain terms which are used in the specification and appended claims.

As used herein the term “inverted phase” with respect to centrifugation means that the less dense or lighter fluid phase is in the portion of the centrifugation tube distal to the axis of rotation (the “bottom”) and the denser or heavier fluid is in the portion of the centrifugation tube proximal to the axis of rotation (the “top”). Because the denser or heavy phase is typically in the distal portion of the tube after centrifugation, the phases are referred to as “inverted” when the light phase is at the distal end and the heavy phase is at the proximal end.

As used herein, the term “heavy phase” refers to a fluid layer in a centrifugation tube which is denser or heavier per unit volume than another phase in the same tube. The terms “heavy” and “denser” are used interchangeably herein. In centrifugation with two fluid phases, the heavy phase is often aqueous, but need not be.

As used herein, the term “light phase” refers to a fluid layer in a centrifugation tube which is less dense or lighter per unit volume than another phase in the same tube. The terms “less dense” and “lighter” are used interchangeably herein. In centrifugation with two fluid phases, the light phase is often organic (e.g., oil), but need not be.

As used herein, the term “aqueous phase” refers to a fluid phase in which water is the primary solvent, although other polar components that are miscible with water may be present. The aqueous phase may also include various solutes and suspended particles. Notably, in the present invention, the aqueous phase may include mitochondria.

As used herein, the term “organic phase” refers to a fluid phase in which a substantially non-polar organic molecule is the primary solvent, although other non-polar components that are miscible with the primary organic solvent may be present. The organic phase may also include various solutes and suspended particles.

As used herein, the term “closed-bottom tube” means any container which may receive fluids and retain them during centrifugation. Closed-bottom tubes may have various shapes, and may be referred to by various terms, including centrifugation tubes, test tubes, sample tubes, sealed tubes, vials, cuvettes, sealed pipettes, etc. A closed-bottom tube may be produced by sealing one end of an open-bottom tube, such as by plugging, crimping or heat-sealing one end of the tube.

As used herein, the term “in vitro fertilization” and the abbreviation “IVF” refer to any assisted reproductive technology in which an oocyte is fertilized outside the body of a female. IVF includes procedures in which oocytes are mixed with sperm in containers such as petri dishes or plates with wells to allow fertilization, with or without removal of the zona pellucida, as well as procedures involving intracytoplasmic sperm injection (ICSI) into oocytes.

As used herein, the terms “biocompatible” and “pharmaceutically acceptable” mean that a substance is not toxic to living cells or organelles at the concentration at which it is present, and is physiologically tolerable and does not produce a severe allergic, pyrogenic or similarly undesired reaction when administered to a mammal.

As used herein, the term “carrier” refers to a diluent, adjuvant, excipient, or vehicle with which a compound is administered. Biocompatible or pharmaceutically acceptable carriers can be sterile liquids, such as water and oils, including those of petroleum, animal, vegetable or synthetic origin, such as mineral oil, vegetable oil and the like. Water or other aqueous solutions, saline solutions, aqueous dextrose and glycerol solutions may be employed as carriers, particularly for injectable solutions. Suitable carriers are described in Remington's Pharmaceutical Sciences, 18th Ed., EW Martin (ed.), Mack Publishing Co., Easton, Pa.

As used herein, the terms “about” or “approximately” mean within ten percent (10%) of the numerical amount cited.

As used herein, the terms “increased” or “decreased” mean at least 10% more or less, respectively, relative to a reference state.

As used herein, unless the context clearly indicates otherwise, the term “a” means one or more.

As used herein, unless the context clearly indicates otherwise, the term “or” means the inclusive and/or and not the exclusive either/or.

### General Considerations

The present invention is dependent, in part, upon the development of methods for collecting and concentrating mitochondria. In some embodiments, the methods provide for the production of concentrated mitochondrial preparations with more intact, viable mitochondria than some prior art methods, which damaged or lost a greater percentage of the mitochondria. In addition, in some embodiments, the methods provide for the production of mitochondrial preparations in which the concentration of mitochondria is greater than achieved in the prior art. In addition, the methods provide for the production of concentrated mitochondrial preparations without tagging the mitochondria with, for example, chromophores, fluorophores, antibodies or other detectable and selectable markers. In addition, the methods provide for the production of concentrated mitochondrial preparations that are substantially free of molecular tags such as chromophores, fluorophores, antibodies or other detectable and selectable markers.

### Inverted Phase Centrifugation

The present invention employs inverted phase centrifugation to produce a concentrated mitochondrial preparation. The methods of the invention include centrifugation of a closed-bottom tube in which there is a heavy phase comprising a mitochondrial suspension in the top of the tube (i.e., the portion of the tube proximal to the axis of rotation), a light phase in the bottom of the tube (i.e., the portion of the tube distal to the axis of rotation), and a phase interface between the heavy phase and the light phase.

The tube is centrifuged at a speed and for a time sufficient to cause mitochondria in the mitochondrial suspension to form a concentrated mitochondrial layer in the heavy phase at the interface between the light phase and the heavy phase. However, the speed and time of centrifugation must be controlled to prevent inversion of the light and heavy phases to their normal configuration (i.e., heavy phase on the bottom and light phase on the top). As will be apparent to one of skill in the art, the likelihood of the phases inverting increases with centrifuge speed (i.e., increasing G force), increasing centrifuge time, increasing centrifuge tube diameter, and increasing differences in the density of the heavy and light phases. Determination of appropriate combinations of speed, time, diameter and density requires only routine experimentation.

In the overall process of producing a concentrated mitochondrial preparation from whole cells, a variety of different values of the relative centrifugal force (RCF) or centrifuge “speed” may be employed. For example, in sedimenting whole cells, the RCF value may be in the range of 500-1,500 G. After lysis, a first sedimentation at an RCF in the range of 500-1,500 G may be employed to sediment larger organelles or membrane fragments without sedimenting the mitochondria. After removing the supernatant, the supernatant can be centrifuged again, this time at an RCF in the range of 5,000-12,500 G (e.g., 7,000-10,000 G) to produce a crude fraction including mitochondria in a pellet. The pellet can be resuspended and sedimented multiple times at RCFs in the range of 5,000-12,500 G to improve the purity of the mitochondrial pellet. Finally, for the inverted-phase centrifugation, the mitochondrial pellet can be resuspended in the heavy phase and centrifuged again at an RCF in the range of 7,500-12,500 G (e.g., 10,000 G) to produce a concentrated mitochondrial pellet at the phase boundary.

After centrifugation, a volume of fluid comprising at least a portion of the concentrated mitochondrial layer is collected to produce the concentrated mitochondrial preparation. The layer can be collected by, for example, pipetting the mitochondrial layer at the interface between the heavy and light phases. Although it would be preferable to collect only the mitochondrial layer, collecting a small amount of the light phase is acceptable for certain applications. For example, as described below, in applications involving mitochondrial transfer to oocytes in conjunction with IVF treatment, the concentrated mitochondrial preparation may be placed on a plate or dish and be covered by an organic fluid layer (e.g., ICSI oil) to prevent evaporation. Thus, in such applications, small amounts of the light phase fluid are likely to separate from the heavy phase fluid and merge into the covering organic fluid.

In some embodiments, the closed-bottom tube is formed during the preparation of the inverted phases for centrifugation. Thus, in some embodiments, the methods of the invention include the steps of drawing a volume of a first fluid comprising a mitochondrial suspension into an open-bottom tube, and then drawing a volume of a second fluid into the tube. The first and second fluids form, respectively, a heavy phase comprising the first fluid and mitochondrial suspension in one portion of the tube, and a light phase comprising the second fluid in another portion of the tube, and define a phase interface between the heavy phase and the light phase. The end of the tube proximal to the light phase is then sealed to form a closed-bottom tube. In some embodiments, the tube is sealed across the light phase (i.e., the seal is created within the light phase such that a portion of the light phase is excluded from the tube).

The closed-bottom tube thus formed can then be placed in the centrifuge with the closed-end distal to the axis of rotation, and can be centrifuged, as described above, to produce the concentrated mitochondrial layer. The concentrated mitochondrial preparation is then obtained by collecting the concentrated mitochondrial layer, as described above.

### Mitochondrial Suspensions and Heavy Phase Fluids

A mitochondrial suspension can be prepared by any standard means known in the art. For example, whole cells (e.g., freshly obtained or thawed frozen cells) can be lysed chemically or mechanically to release the cytoplasm, and the crude lysate can be regarded as a mitochondrial suspension. Preferably, however, the crude lysate is further processed to purify and the preparation before employing the phase inverted centrifugation of the invention.

Prior art methods of mitochondrial isolation can be found in, for example, Tzeng et al. (2004), Fertil. Steril. 82(S2), S53; Yamaguchi et al. (2007), Cell Death Differ. 14:616-624; and Shufaro et al. (2012), Fertil. Steril. 98(1):166-72. These methods can be employed to produce a first mitochondrial suspension, and then the inverted-phase centrifugation method of the invention can be applied. Alternatively, the mitochondrial suspension can be prepared substantially as described in the examples below.

In some embodiments, the mitochondrial suspension is an aqueous suspension. In some embodiments, the aqueous suspension includes non-pyrogenic, high purity grade water produced by, for example, water for injection (WFI) which has passed mouse embryo testing (MEA) (e.g., Charles River Laboratories, Wilmington, Mass.) or the equivalent (e.g., Water for Assisted Reproductive Technologies (A.R.T.), Irvine Scientific USA, Santa Ana, Calif.) or other biocompatible or pharmaceutically acceptable carriers (e.g., dextrose, sucrose, glycerol, citrate).

In some embodiments, the mitochondrial suspension is produced from isolated oogonial stem cells (OSCs). OSCs can be obtained as described, for example, in WO 2005/121321, U.S. Pat. No. 7,955,846 and U.S. Pat. No. 8,652,840.

### Centrifugation Tubes

The centrifugation tubes of the invention can be any closed-bottom tube with a sufficiently small inner diameter to prevent inversion of the phases during centrifugation. In some embodiments, the closed-bottom tube is a pipette or a micropipette or collection tube which has been sealed at one end (before or after loading the heavy and light phases).

The tubes are capable of holding liquid volumes of at least 1 μl, 5 μl, 10 μl, 20 μl, 30 μl, 40 μl or 50 μl, and may holder larger volumes up to 100 μl, 150 μl, 200 μl, 250 μl, 300 μl, 500 μl or more.

In some embodiments, the tube is has an inner diameter (measured at the phase boundary) from 1 μm, 10 μm, 20 μm, 40 μm, 50 μm 60 μm, 70 μm, 80 μm, 90 μm or 100 μm, up to 150 μm or more. In some embodiments, the inner diameter (measured at the phase boundary) is 25-250 μm, 50-150 μm, or 75-100 μm. Note that the inner diameter need not be constant, and that tapered tubes are contemplated for use in the invention (e.g., 80 μm Flexipet® micropipettes (Cook Medical, Bloomington, Ind.) described in the examples below).

The tubes can be made of glass, plastic and/or any semi-flexible tubing. As used herein, the term “plastic” means any of various synthetic or semi-synthetic organic solids produced by polymerization that are capable of being molded, extruded, or cast into shapes and films. Plastic materials include, but are not limited to, various including polyethylenes (PE), high-density polyethylenes (HDPE), low-density polyethylenes (LDPE), polyethylene terephthalates (PET), polypropylenes (PP), polyvinyl chlorides (PVC), polyvinylidene chlorides (PVDC), polystyrenes (PS), high impact polystyrene (HIPS), acrylonitrile butadiene styrenes, polyamides, polycarbonates, polyurethanes and nylons. In some embodiments, for example, the closed-bottom tubes are formed by sealing the bottom of a polycarbonate pipette (e.g., Flexipet® micropipettes, Cook Medical, Bloomington, Ind.) or glass pipettes (e.g., Drummond Scientific Company, Broomall, Pa.).

In some embodiments, the closed-bottom tube may be placed within one or more other tubes, sleeves, straws, holders or adaptors (e.g., a centrifuge tube) to hold and/or cushion it within the rotor assembly of a centrifuge.

### Light Phase Fluids

The light phase fluids of the invention may comprise one or more of various organic compounds with the requirement that the light phase is capable of forming a phase boundary with the heavy phase which prevents mitochondria from entering the light phase at the centrifuge speed employed. In some embodiments, the light phase fluids comprises one or more biocompatible oils including, but not limited to, mineral oil (e.g., CAS 8042-47-5, Sigma Aldrich, St. Louis, Mo.; SAGE Media Oil for Tissue Culture, Catalog # ART-4008, ORIGIO A/S, M∪l∘v, Denmark), light oil, paraffin oil, and any other in vitro fertilization (IVF) culture oil having received marketing approval (e.g., 510(k) acceptance by the U.S. FDA (e.g., ICSI oils). Other organic fluids with higher osmolality than isotonic solutions, which can be used alone or in combination include, but are not limited to, polyvinylpyrrolidone (PVP) solutions, sucrose and sucrose polymer (e.g., Ficoll®, Sigma-Aldrich, St. Louis, Mo.) solutions, colloidal silica particle (e.g., Percoll®, Sigma-Aldrich, St. Louis, Mo.) solutions, and the like.

In some embodiments, the light phase fluid has an osmolality of over 250 OsM.

### Concentrated Mitochondrial Preparations

In another aspect, the invention provides concentrated mitochondrial preparations produced according to the methods of the invention. In some embodiments, the mitochondrial in the concentrated mitochondrial preparation has a concentration of at least one mitochondrion per picoliter. In some embodiments, in the disclosed preparations, the solution includes between 1 and 10,000 mitochondria per picoliter; between 50 and 10,000 mitochondria per picoliter; between 100 and 10,000 mitochondria per picoliter; between 150 and 10,000 mitochondria per picoliter; between 200 and 10,000 mitochondria per picoliter; between 250 and 10,000 mitochondria per picoliter; between 300 and 100,000 mitochondria per picoliter; between 400 and 10,000 mitochondria per picoliter; between 450 and 10,000 mitochondria per picoliter; and between 500 and 10,000 mitochondria per picoliter. In some embodiments, at least 90%, 91%, 92%, 93%, 94% 95%, 96%, 97%, 98%, 99%, or 99.5% of the mitochondria in the concentrated mitochondrial preparation are functional.

“Functional mitochondria” refers to mitochondria that are biologically active. The percent of functional mitochondrial in a concentrated mitochondrial preparation can be determined by using methods known in the art, including by oxidative phosphorylation (OXPHOS) ATP assay, measuring cytochrome C release, measuring respiration rate, and/or by visual inspection. In some embodiments, mitochondrial analysis is performed on the concentrated mitochondrial layer to determine the percent of functional mitochondria.

Visual inspection can include the use of far-field fluorescence microscopy; various microscopic techniques and specific fluorescent probes can be used to stain mitochondria, including the use of green fluorescent protein (GFP), nanoscopy or super-resolution fluorescence, high-resolution electron microscopy and electron tomography. Marin-Garcia (2013), “Methods to Study Mitochondrial Structure and Function,” in Mitochondria and Their Role in Cardiovascular Disease, Springer US, New York.

Various in vitro approaches to assessing mitochondrial function include spectrophotometric enzyme assays, bioluminescent measurement of ATP production, and polarographic measurement of oxygen consumption. Electrophoretic techniques can be used to assess mitochondrial function. For example, two-dimensional polyacrylamide gel electrophoresis followed by Western immunoblotting can be used to analyze the content of mitochondrial proteins. Highly specific and sensitive immunodetection methods enhance the use of electrophoresis techniques. Various specific antibodies to mitochondrial proteins can be used in both immunocytochemical and Western immunoblot analysis. These antibodies include ATP synthase subunits, cytochrome c and cytochrome c oxidase, PGC-1, and mtTFA.

The following examples illustrate some preferred modes of practicing the present invention, but are not intended to limit the scope of the claimed invention. Alternative materials and methods may be utilized to obtain similar results.

### Example 1

### Producing a Mitochondrial Suspension

### Tissue Dissociation

Tissue from a frozen ovarian biopsy was used as a source of mitochondria. Tissue dissociation procedures known in the art were performed. All steps were performed in a biosafety cabinet, except cell sorting, using appropriate aseptic techniques well-known in the art.

Three (3) four-well plates were obtained. On each of the four-well plates, three of the wells were labeled as “1”, “2”, and “3.” One plate was designated per piece of source material. Using a 1000 μL micropipette, cryopreservation medium and holding medium were added to each well on each plate per volumes in the table below, and were gently mixed by pipetting up and down three to five times.

| Medium | Well 1 | Well 2 | Well 3 |
|---|---|---|---|
| Cryopreservation | 600 μL | 300 μL | 0 μL |
| Holding | 300 μL | 600 μL | 900 μL |

The following materials were prepared: one bottle of 3.636 KU/mL DNASE I; one bottle of 400 U/mL Collagenase (2000MTF from Liberase MTF C/T kit; Roche Diagnostics, Indianapolis, Ind.); and one bottle of 1 mg/mL Thermolysin (from Liberase MTF C/T kit; Roche Diagnostics, Indianapolis, Ind.). A C-tube labeled “BSS/DNASE/Liberase solution” was prepared using the specifications in the following table:

| | HBSS with | Reconstituted | Reconstituted | Reconstituted |
|---|---|---|---|---|
| Compo- | MgCl₂/ | DNASE I | Thermolysin | Collagenase |
| nent | CaCl₂ | (3.636 KU/mL) | (1 mg/mL) | (400 U/mL) |
| Amount | 9.0 mL | 27 μL | 27 μL | 27 μL |

The tube was placed in 37±1° C. water bath until further use. A solution of 10 mL of HBSS without CaCl₂ and MgCl₂ was aliquoted into a 50 mL conical tube and placed in a 37±1° C. water bath until further use.

An appropriate number of vials was submerged in liquid nitrogen. All tissue vials were thawed simultaneously by air thawing for 60 seconds at ambient temperature. After the 60-second air thaw, the vials were incubated simultaneously in a 37.0° C.±1.0° C. water bath for 120 seconds. The vials were sanitized. Using sterile forceps, each piece of tissue source material was transferred from vials into well #1 of each of the previously-prepared 4-well culture plates. One piece of tissue was placed in each plate and completely submerged in the medium, and the source material was held in each labeled well #1 for 10 minutes. The source material was transferred from each labeled well #1 to each labeled well #2, was completely submerged in the medium, and was held in each of the labeled well #2 for 10 minutes. The source material was transferred from each labeled well #2 to each labeled well #3, completely submerged in the medium, and was held in each labeled well #3 for 5 minutes. All pieces of source material were transferred to the C-tube containing the HBSS/DNASE/Liberase solution.

The C-tube was transferred to a tissue dissociator (GentleMACS™, Miltenyi Biotec, Bergisch Gladbach, DE) and the tissue was dissociated according to manufacturer's instructions.

### Cell Sorting Procedure

In order to obtain cells with a high percentage of functional mitochondria from the ovarian tissue sample, antibodies against the VASA protein were used to sort oogonial stem cells (OSCs) from other cells present in the ovarian biopsy tissue.

Using a glass 5 mL serological pipette, the dissociated cell mixture was transferred to a 70 μm cell strainer, which was placed onto a 50 mL conical tube. A C-tube was rinsed with 4 mL of warmed HBSS without CaCl₂ and MgCl₂. The rinse volume was passed through the strainers. Next, 6.75 mL of cell suspension from the 50 mL conical tube was transferred to a 15 mL conical tube labeled “Filtered Cells 1 of 2,” and an equal volume was placed in a 15 mL conical tube labeled “Filtered Cells 2 of 2.” The tubes were centrifuged at 1200 relative centrifugal force (RCF) for 10 minutes at ambient temperature (range: 18.0-22.0° C.), with the brake off (0). The bulk of the supernatant was removed and transferred to a 50 mL conical tube labeled “First Supernatant.” Any residual volume (approximately 500 μL) was removed with a micropipette from each tube.

The pellet in Filtered Cells 1 of 2 was re-suspended using 250 μL of 2% block solution. The cell suspension was transferred to the pellet in Filtered Cells 2 of 2 tube. The Filtered Cells 1 of 2 tube was rinsed using 250 μL of 2% block solution. The rinse volume was transferred to the Filtered Cells 1 of 2 tube, and the pellet was re-suspended. The cell suspension was observed for visible clumps. If clumps were visible after pipetting, the cells were re-filtered as needed.

The volume in the Filtered Cells 2 of 2 tube was measured and recorded. A 2404 volume of 1% wash solution was added to a 1.5 mL microfuge tube labeled “Cell Count.” The cell suspension was re-suspended, and 10 μL of cells was transferred to the 1.5 mL Cell Count tube.

Cell sorting was performed using the Fluorescence-Activated Cell Sorting (FACS). A 500 μL volume of 1% wash solution was used to clear the lines. The Cell Count tube was placed in the sample loader, and the sample was acquired for approximately two minutes. The cell number was calculated using methods known in the art. If total cell number was greater than 7×10⁶ cells, steps were performed to divide the sample in half, such that half the sample was cryopreserved, and the remaining sample was stained and used for cell sorting (see below). If total cell number was less than or equal to 7×10⁶ cells, then the cells were incubated at room temperature for 20 minutes with 2% block solution.

Primary antibody aliquots were thawed and centrifuged at 200×g (RCF) for one minute at ambient temperature (20° C.±2.0° C.), with the brake set on high (9) to ensure volume is contained at the bottom of vial. A volume of 2 μL was removed from the re-suspended cell pellet (“first pellet”), and transferred to a new 15 mL conical tube labeled “Negative Control.” A volume of 5 mL of 1% wash solution was added to the Negative Control tube.

The calculated volume of antibody was added to the first pellet tube and mixed well. The tube was incubated for 15 minutes at room temperature in the dark. A volume of 5 ml of 1% wash solution was added to a 15 ml tube labeled “Antibody Labeled” cells. The Antibody Labeled cells and the Negative Control were both centrifuged at 1200×g (RCF) for 5 minutes at ambient temperature (20.0° C.±2.0° C.), with the brake set on high (9).

Tubes containing “Antibody Labeled Cells First Supernatant” and “Negative Control Supernatant” were prepared. Using a micropipette, supernatant was removed from the 15 mL conical tube containing Antibody Labeled cells and transferred to the appropriately labeled 15 mL conical tube. The Antibody Labeled cells were re-suspended using 3754, of 1% wash solution. The cell suspension was transferred to a 1.5 mL microfuge tube labeled as “Antibody Stained Cells.” The Antibody Stained Cells were re-suspended. The cell suspension was observed and, if clumps were visible, the cells were re-filtered using a strainer as needed.

Using a micropipette, supernatant was removed from the Negative Control conical tube and transferred to the appropriately labeled 15 mL conical tube. The cell pellet in the Negative Control tube was re-suspended using 500 μL of 1% wash solution and transferred to a 1.5 mL microfuge tube labeled Negative Control.

A volume of 500 μL of 1% wash buffer was aliquoted into a new 15 mL conical tube labeled “VASA+Cells”. A volume of 500 μL of 1% wash buffer was aliquoted into another 15 mL conical tube labeled “Rinse 2.” The “Negative Control”, “Antibody Stained Cells”, “Rinse 2” and “VASA+Cells” tubes were used for cell sorting.

Cell sorting was performed using a Sony FACS machine with the recording rule fields set so that the Event limit was 10,000. Two empty 15 mL conical tubes were placed in the sort collection holder, and the left and right sort gates were set to “Cells.” The stopping limit was set to 500 events. The Negative Control cells were re-suspended and loaded in the sample loading area. The event rate was adjusted by altering the Sample Pressure to achieve between 200-2000 events per second. Minor adjustments were made in the Alexa Fluor 647 detector settings to ensure events were between 10² and 10³. The cells gate were adjusted to encompass approximately 90% of the events in the back-scatter (BSC) versus forward-scatter (FSC) plot. The VASA+ gate (reflecting the positively-stained cells) was adjusted as necessary to not include any of the unstained population.

The Negative Control sample was removed from the sample tube holder and put aside. As needed, chip alignment and sort calibration and performed again, followed by probe and sample line sterilization. The 15 mL conical tubes were removed, and the VASA+ tubes were inserted with 1% wash solution into the left sorting position of the sort chamber. The record rule fields were set so that the event count was 2,000 and the sample stop condition was “Recording.” The gates were each set to “All Events.” Pressure was varied to maintain 1500-2000 counts per reading. The Rinse 2 tube was loaded onto the sample tube holder, and the lines were cleared with wash solution. The Antibody Stained Cells tube was re-suspended and loaded onto the sample tube holder. The Antibody Stained Cells were acquired, and the events were registered. It was confirmed that the events were within the Alexa Fluor 647 detectors by observing the dot plot (AF647 versus BSC). Data acquisition was confirmed. The Antibody Stained Cells were removed from the loading area and stored in an area protected from light.

The stained cell population was evaluated. Populations representing specific and non-specifically-stained cell populations were visible in the dot plot (AF647 vs BSC). Adjustments were made to ensure that the positive population was visible. The VASA+ gate was migrated along the X-axis (AF 647) to encompass main higher intensity stained cell population of the pre-sort sample gate.

The recording rule event limit was changed to 10,000,000. The sort limit was changed to zero (no stopping limit), and the sort mode was changed to “Yield.” The sort gate on the left was selected as VASA+ gate. It was ensured that the VASA+ cells tube was placed on the left side of the collection holder. The lines were cleared with “Rinse 2” wash solution. The cells in the Antibody Stained Cells tube were re-suspended, the tube was loaded onto the sample tube holder, and the cells were sorted until the sample tube was fully depleted. During the sort, the pressure was adjusted to keep the event rate at 200-2000 events per second. The 15 mL FACS tube containing sorted VASA+ fraction was removed, and the volume was measured. The sides of the 15 mL tube were rinsed with 1000 μL of 1% wash solution.

OSC freezing was performed using the following steps. A cryopreservation controller (Crysalys™ PTC-9500, Biostasys, Inc., NV) was connected to the cryopreservation chamber. The covered cryo chamber was placed into the liquid nitrogen bath. Additional liquid nitrogen was slowly added to increase the volume until liquid nitrogen was approximately 3 cm from the top of the cryo chamber. The program had the following settings: start at 18° C.; Final temperature of −80° C., decreasing at a rate of 1° C./minute; hold at −80° C. for 10 minutes; and signal end of run at −80° C.

The sorted VASA+ cell fraction was sorted at 1200 RCF for 10 minutes at 5° C. (range: 2-8° C.), with the brake set on medium (5). If the tissue sample was divided prior to cell sorting, the cryovial containing pre-sorted ovarian cells was obtained from 2-8° C. storage and centrifuged with VASA+ cells. Supernatant was removed, and the cell pellet was re-suspended with 500 μL of cold cryopreservation media. Using a 1000 μL micropipette, the supernatant from the 15 mL conical tube was removed and transferred to a 15 mL conical labeled “First VASA+Supernatant.” Any remaining volume was removed using a 200 μL pipette without disturbing the pellet(s). The pellet was re-suspended in 500 μL of cold cryopreservation media, and the cell suspension is transferred to the cryovial. The cryovial was placed in the cryo chamber. After the program finished, the cryovial was stored in a dewar full of liquid nitrogen.

### Mitochondria Isolation Procedure

The mitochondria concentration procedure was performed as follows. A 20 mL volume of 1× isolation buffer was prepared based on the specifications listed in the table below:

| Component | Isolation Buffer | 10% HSA | Water |
|---|---|---|---|
| Concentration | 1X | 2% | 1X |
| Volume (mL) | 10.0 | 1.0 | 9.0 |

The components were added into a 50 mL conical tube, mixed, and 2-3 mL was aliquoted into separate conical tubes for re-suspension and rinses. The tubes were labeled “1× Isolation Buffer” and stored in the refrigerator or metallic thermal beads (Lab Armor™ Beads, Lab Armor LLC, Cornelius, Oreg.). Similarly, tubes of “1% HSA in HBSS” were prepared. A volume of 10 mL of 1% HSA in HBSS was prepared and 2-3 mL was aliquoted into separate conical tubes for re-suspension and rinses. The tubes were labeled “1% HSA in HBSS” and stored in the refrigerator or metallic thermal beads.

The cryopreserved vials of human OSCs containing the sorted VASA+ fraction were transferred to a 2 L dewar flask, and the vials were submerged in liquid nitrogen. The vials were air thawed for 60 seconds in ambient temperature. After the 60-second air thaw, the vials were incubated in a 37.0° C.±1.0° C. water bath for 120 seconds. Using a 1000 μL micropipette, the entire volume from the vial was slowly transferred into a 1.5 mL microcentrifuge tube labeled “Thawed VASA+Fraction.” The cryovial was rinsed with 500 μL of 1% HSA in HBSS using a 1000 μL serological pipette; the rinse was transferred into the 1.5 mL microcentrifuge tube. The VASA+ fraction pellet was centrifuged at 1200×G (RCF) for 10 minutes at 5.0° C. (range: 2.0-8.0° C.), with the brake set on high (9). Upon completion of the spin, the tube was transferred into a biosafety cabinet.

Using a 1000 μL micropipette, supernatant from the 1.5 mL microcentrifuge tube was carefully removed and transferred to a 15 mL conical tube labeled “First VASA+Supernatant.” Any remaining volume was removed without disturbing the pellet. The pellet was re-suspended gently in 150 μL of cold 1× isolation buffer, and re-suspended. The 1.5 mL microcentrifuge tube containing the cell suspension was placed into a bench top cooler module (MiniFridge II™, Boekel Scientific, Feasterville, Pa.) or metallic thermal beads.

A 17-18 mL aliquot of 1× isolation buffer was obtained using a 20 mL syringe, and the syringe was attached to the transfer line-cannula assembly. The cannula was submerged into 1× isolation buffer. The syringe assembly was loaded onto a syringe pump (Harvard Apparatus, Holliston, Mass.) while keeping the cannula submerged in the 1× isolation buffer. A second empty 20 mL syringe was obtained, and approximately 5 mL of air was pulled to balance the pump. Using the pump, the remaining buffer in the syringe was flushed out into a 1× isolation buffer container. Using the pump, 1.5 mL of cold 1× isolation buffer was withdrawn into the syringe, with the following pump parameters:
Pump Mode: Withdraw;
Flow Rate (μL/sec): Max 75;
Programmed Vol. (μL): 1500; and
Diameter/Syringe Size: 20.05 mm/20 cc.

A pump program was selected with the parameters listed in the table below:

| | | Flow Rate | Programmed | Cy- | Diameter/ |
|---|---|---|---|---|---|
| Step | Pump Mode | (μL/sec) | Vol. (μL) | cles | Syringe Size |
| 1 | Withdraw | Max 75 | 150 | | 20.05 mm/ |
| | | | | | 20 cc |
| 2 | Delay - | | | | |
| | 5 seconds | | | | |
| 3 | Autofill | Infuse: 1000 | 400 | 40 | 20.05 mm/ |
| | (Infuse/ | Withdraw: 25 | | | 20 cc |
| | Withdraw) | | | | |
| 4 | Infuse | 1000 | 850 | | 20.05 mm/ |
| | | | | | 20 cc |

The cannula was placed into the cell suspension so that it touched the bottom of the tube. If the cells settled to the bottom of the tube, then the cell suspension was mixed gently with a 200 μL pipette as needed. The pump was started to withdraw the cell suspension into the syringe. The cannula was transferred to a clean and cold 1.5 mL microcentrifuge tube.

A second pump program was selected with the parameters listed in the table below:

| | | Flow Rate | Programmed | Diameter/ |
|---|---|---|---|---|
| Step | Pump Mode | (μL/sec) | Vol. (μL) | Syringe Size |
| 1 | Infuse | 1000 | 800 | 20.05 mm/ |
| | | | | 20 cc |

Once the pump program ended, the syringe was removed from the pump and disconnected from the transfer tubing. The cannula (still connected to the transfer tubing) was left in the cold 1.5 mL microcentrifuge tube. The syringe, tubing, and cannula were properly discarded.

Both 1.5 mL microcentrifuge tubes had an approximately equal volumes of lysate. The two lysate tubes (“Lysate 1” and “Lysate 2” were centrifuged at 800×G (RCF) for 10 minutes at 5.0° C. (range 2.0-8.0° C.), with the brake set on medium (5). Using a 200 μL pipette, equal volumes of supernatant from the “Lysate 2” tube was transferred into tubes labeled “Supernatant 1” and “Supernatant 2.” The two supernatant tubes were centrifuged at 7000×g (RCF) for 30 minutes at 5±3° C. (range: 2-8° C.), with the brake set on medium (5). Upon completion of spin, using a 1000 μL pipette, supernatant was removed from both tubes to a 15 mL conical tube labeled “Mito High Speed Spin Supernatant 1.” Any residual supernatant was removed using a 200 μL pipette tip without disturbing the pellet. The pellet was gently re-suspended using a 200 μL pipette in the Supernatant 1 tube with 100 μL of cold 1× isolation buffer. Using the same pipette tip, the suspension was transferred to the Supernatant 2 tube. The Supernatant 1 tube was rinsed 5-10 times with 100 μL of cold 1× isolation buffer, and the solution was transferred to the Supernatant 2 tube. The pellet in the Supernatant 2 tube was re-suspended, and the empty Supernatant 1 tube was discarded. The Supernatant 2 tube was centrifuged at 7000×g (RCF) for 30 minutes at 5.0° C. (range: 2.0-8.0° C.), with the brake set on medium (5). That tube was re-labeled “Mito High Speed Spin Supernatant 2.” Additional centrifugation was performed if the pellet did not adhere to the wall of the tube and/or supernatant was not removed. The Supernatant 2 tube was, if applicable, centrifuged at 700×g (RCF) for 10 minutes at 5.0° C. (range: 2.0-8.0° C.), with the brake set on medium (5). Using a 200 μL pipette, the supernatant was gently removed to a tube labeled “Mito High Speed Spin Supernatant 3.”

Using a 20 μL micropipette, the pellet was gently re-suspended in 6 μL of cold respiration buffer. The buffer was pipetted up and down 5-10 times to rinse the pellet. The volume in the microcentrifuge tube was measured and transferred to the cold 0.5 mL cryovial labeled as “Mitochondrial Suspension.” A volume of 1 μL of suspension was transferred into a 0.5 mL cryovial for quantitative polymerase chain reaction (qPCR) analysis. The qPCR analysis confirmed the presence of mitochondria from the isolated OSCs.

### Example 2

### Concentrated Mitochondrial Preparation

### Materials and Equipment

As described in Example 1, the mitochondrial solution was an autologous mitochondria formulation obtained by extraction from isolated OSCs, which are also known as egg precursor cells or female germline stem cells. The mitochondria solution was held at 5° C.±3° C. until further processed.

The concentrated mitochondrial preparation produced using the methods described herein were used for various applications, including but not limited to AUGMENT^{SM} and IVF methods. IVF Sites used site-specific standard IVF equipment, materials and reagents throughout the IVF process. Additional materials were supplied by OvaScience, Inc. including but not limited to:

| Material | Source | Product Number |
|---|---|---|
| Micro Centrifuge | Thermo Fischer | 75-002-446 |
| | Scientific or equivalent | |
| Centrifuge Rotor | Thermo Fischer | 05-375-105 |
| | Scientific or equivalent | |
| Flexipet, 80 μm | Cook Medical or equivalent | G26181 |
| Flexipet, 600 μm | Cook Medical or equivalent | G26057 |
| Cryo Bio System | Biogenics, Inc. or equivalent | 006433 |
| (CBS) Straw, 133 mm | | |

Materials and equipment for use in clinical processing and AUGMENT^{SM} processing are known in the art.
Mitochondrial Preparation with Inverted (Aqueous) Heavy and (Organic) Light Layers

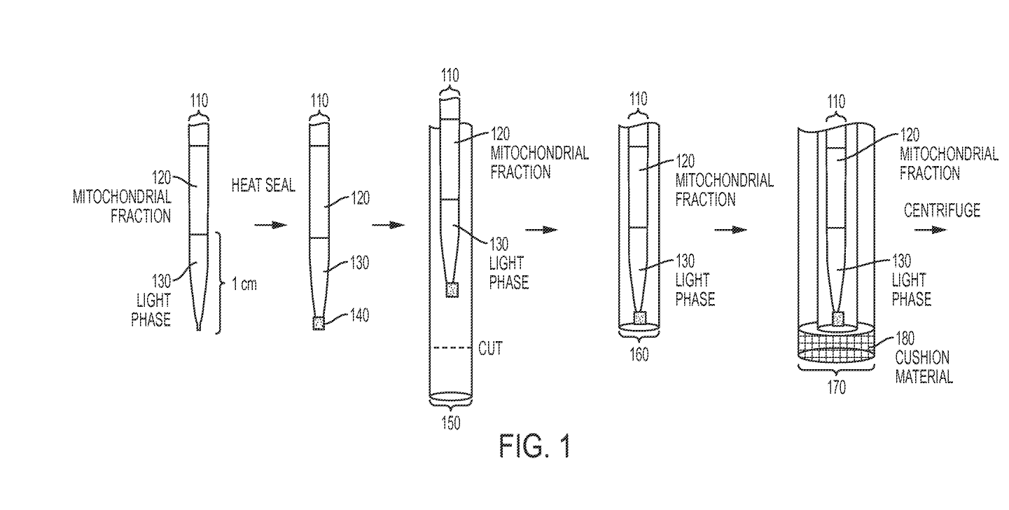

Tubes loaded with mitochondrial solution were prepared. In some embodiments, the tube is a plastic tube. Using a 10 μl pipette, mitochondrial solution prepared in Example 1 was aspirated and withdrawn approximately 30 times to re-suspend the solution in the vial. Using a pipette, 2 μl of the mitochondria suspension was removed and dispensed onto an empty Intracytoplasmic Sperm Injection (ICSI) plate. The vial containing mitochondrial suspension was maintained at 5° C.±3° C. As shown in FIG. 1, the dispensed 2 μl of the mitochondria suspension 120 was loaded into a 80 μm flexible micropipette (80 μm Flexipet®, Cook Medical, Bloomington, Ind.), depicted as tube 110. In some embodiments, the size of the flexible micropipette ranges from 50 μm to 140 μm. In some embodiments, various pipette materials can be used, including glass, semi-flexible tubing, and plastic. The layer 120 in FIG. 1 is an aqueous layer that contains the mitochondrial suspension in the proximal portion of the tube.

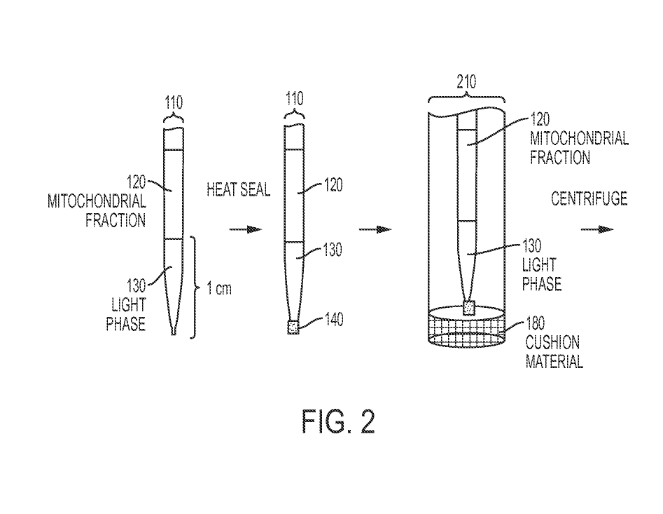

Next, about 1 cm of an organic light phase was drawn into the tube 110. The layer 130 in FIG. 1 is the light phase that contains an organic fluid in the distal portion of the tube. In this example, the organic fluid was a biocompatible oil, specifically an ICSI oil. In other embodiments, the organic fluid can be a mineral oil, paraffin oil, light oil, IVF culture mineral oil, PVP solution, or sucrose solution, etc., as described above. As shown in FIG. 1, in some embodiments, the light phase end of the filled 80 μm tube was heat sealed such that the open-ended tube became a closed-bottom tube, as shown by the seal 140. The heat seal was repeated as needed, and the integrity of the seal was verified by microscopic observation. To ensure a snug fit into the centrifuge, a larger tube 150 (e.g., a 600 μm Flexipet) was slid over the heat-sealed end of tube 110 (e.g., the 80 μm Flexipet). The excess length of the larger tube 150 that extended beyond the end of the tube 110 (e.g., the 80 μm Flexipet) was removed, as shown by tube 160 that has been cut. The tube 160 was slid into a “cryopreservation straw” 170 so that the larger tube 160 touched the straw's cushion material 180 (e.g., cotton plug). The cryopreservation straw 170 containing the entire assembly, including the mitochondrial suspension in tube 110 was placed in the microcentrifuge at 4° C. The bore of the closed-bottom tube 110 prevents or reduces inversion of the aqueous layer 120 and the organic layer 130. In other embodiments, as shown in FIG. 2, the tube 110 with the aqueous layer 120 and organic layer 130 is fitted into an appropriately-sized tube 210 that fits snugly into a centrifuge.

Universal precautions known in the art were used in these procedures. These precautions are methods of infection control in which all human blood, certain bodily fluids, as well as fresh tissues and cells of human origin are handled as if they are known to be infected with HIV, HBV, and/or other blood-borne pathogens.

### Mitochondrial Centrifugation

The tubes of mitochondrial preparation, as depicted, for example, as tube 170 in FIG. 1 and tube 210 in FIG. 2, were centrifuged at 10,000×G for 20 minutes at 4° C. to produce a concentrated mitochondrial layer at the interface between the heavy phase 120 and the light phase 130. In some embodiments, the tube is centrifuged at a rate of less than 10,000 G. In some embodiments, the tube is centrifuged at a rate between 1,000-5,000 G, 2,500-7,500 G, 5,000-10,000 or 7,500-12,500 G. In some embodiments, the RCF value of centrifugation permit shorter centrifugations times, e.g., 1-2 minutes, 2-4 minutes, 5-10 minutes or 10-15 minutes. Conversely, lower RCF values may require longer centrifugations times.

The speed and time of centrifugation allowed formation of the concentrated mitochondrial layer without inversion of the aqueous and oil layers. After the 20 minutes of centrifugation at 10,000 G was completed, the straw was removed from the centrifuge, and the tube 110 was slid out of the straw 170 and cut tube 160. The concentrated mitochondrial layer was placed in an ICSI plate and overlaid with ICSI oil. The concentrated mitochondrial layer was maintained at a temperature of 5° C.±3° C. for use in AUGMENT^{SM}. These steps were repeated to produce additional concentrated mitochondrial layers.

In some embodiments, the concentrated mitochondrial preparation was used to perform ICSI using methods known in the art. ICSI was performed by adding approximately 1 pL of concentrated mitochondrial preparation during each metaphase II oocyte microinjection. In some embodiments, ICSI was performed using site practices with the addition of approximately 1-3 pL of concentrated mitochondria during each metaphase II oocyte microinjection. For ICSI, the concentrated mitochondrial preparation pellet used ranged from 10 nL-500 nL. In some embodiments, the concentrated mitochondrial preparation has a mitochondrial concentration of between 1 and 10,000 mitochondria per picoliter. In some embodiments, in the disclosed preparations, the solution includes between 1 and 10,000 mitochondria per picoliter; between 50 and 10,000 mitochondria per picoliter; between 100 and 10,000 mitochondria per picoliter; between 150 and 10,000 mitochondria per picoliter; between 200 and 10,000 mitochondria per picoliter; between 250 and 10,000 mitochondria per picoliter; between 300 and 100,000 mitochondria per picoliter; between 400 and 10,000 mitochondria per picoliter; between 450 and 10,000 mitochondria per picoliter; and between 500 and 10,000 mitochondria per picoliter. In some embodiments, the tube 110 has an inner diameter at the interface between the aqueous layer and the organic layer of less than 1 μm. In some embodiments, at least 90%, 91%, 92%, 93%, 94% 95%, 96%, 97%, 98%, 99%, or 99.5% of the mitochondria in the concentrated mitochondrial preparation are recovered functional as determined by qPCR As discussed herein, in some embodiments, the concentrated mitochondria preparations are used in various fertilization methods, including the ICSI and AUGMENT methods.

### EQUIVALENTS

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize, or be able to ascertain using no more than routine experimentation, many equivalents to the specific embodiments of the invention described specifically herein. Such equivalents are intended to be encompassed in the scope of the appended claims.

What is claimed is:

1. A method for producing a concentrated mitochondrial preparation comprising the steps of:
(a) centrifuging a closed-bottom tube containing:
(i) a heavy phase comprising a mitochondrial suspension in the portion of the tube proximal to the axis of rotation;
(ii) a light phase in the portion of the tube distal to the axis of rotation; and
(iii) a phase interface between the heavy phase and the light phase;
wherein the closed-bottom tube is centrifuged at a speed and for a time sufficient to cause mitochondria in the mitochondrial suspension to form a concentrated mitochondrial layer in the heavy phase at the interface between the light phase and the heavy phase, and without causing inversion of the light and heavy phases; and
(b) collecting a volume of fluid comprising at least a portion of the concentrated mitochondrial layer to produce the concentrated mitochondrial preparation.

2. The method of claim 1 wherein the heavy phase is an aqueous phase.

3. The method of claim 2 wherein the heavy phase is obtained by chemical, mechanical or sonic lysis of cells, followed by centrifugation.

4. The method of claim 2 wherein the light phase is an organic phase.

5. The method of claim 4 wherein the light phase comprises a biocompatible oil.

6. The method of claim 5 wherein the biocompatible oil light is selected from the group consisting of a mineral oil, a paraffin oil, a light oil, a cell culture oil and an ICSI oil.

7. The method of claim 2 wherein the concentrated mitochondrial preparation has a concentration of at least one mitochondrion per picoliter.

8. The method of claim 2 wherein the tube has an inner diameter at the interface between the heavy phase and the light phase of less than 1 μm.

9. The method of claim 2 wherein the tube is centrifuged at a rate of less than 10,000 G.

10. The method of claim 2 wherein after drawing the volume of the second fluid into the open-bottom tube, the portion of the tube proximal to the light phase is heat sealed across the portion containing the light phase to form a closed-bottom tube.

11. The method of claim 2 wherein the tube comprises a material selected from the group consisting of polyethylenes (PE), high-density polyethylenes (HDPE), low-density polyethylenes (LDPE), polyethylene terephthalates (PET), polypropylenes (PP), polyvinyl chlorides (PVC), polyvinylidene chlorides (PVDC), polystyrenes (PS), high impact polystyrene (HIPS), acrylonitrile butadiene styrenes, polyamides, polycarbonates, polyurethanes and nylons.

12. The method of claim 2 wherein at least 90% of the mitochondria in the concentrated mitochondrial preparation are functional.

13. A method for producing a concentrated mitochondrial preparation comprising the steps of:
(a) drawing a volume of a first fluid comprising a mitochondrial suspension into an open-bottom tube;
(b) drawing a volume of a second fluid into the tube, wherein the second fluid is less dense than the first fluid, thereby forming a heavy phase comprising the first fluid and mitochondrial suspension in one portion of the tube, a light phase comprising the second fluid in another portion of the tube, and a phase interface between the heavy phase and the light phase;
(c) sealing the end of the tube proximal to the light phase to form a closed-bottom tube;
(d) placing the closed-bottom tube in the centrifuge with the sealed end distal to the axis of rotation;
(e) centrifuging the closed-bottom tube at a speed and for a time sufficient to cause mitochondria in the mitochondrial suspension to form a concentrated mitochondrial layer in the heavy phase at the interface between the light phase and the heavy phase, and without causing inversion of the light and heavy phases; and
(f) collecting a volume of fluid comprising at least a portion of the concentrated mitochondrial layer to produce the concentrated mitochondrial preparation.

14. The method of claim 13 wherein the first fluid is drawn into the tube before the second fluid is drawn into the tube.

15. The method of claim 14 wherein the heavy phase is an aqueous phase.

16. The method of claim 15 wherein the heavy phase is obtained by chemical, mechanical or sonic lysis of cells, followed by centrifugation.

17. The method of claim 15 wherein the light phase is an organic phase.

18. The method of claim 17 wherein the light phase comprises a biocompatible oil.

19. The method of claim 18 wherein the biocompatible oil light is selected from the group consisting of a mineral oil, a paraffin oil, a light oil, a cell culture oil and an ICSI oil.

20. The method of claim 15 wherein the concentrated mitochondrial preparation has a concentration of at least one mitochondrion per picoliter.

21. The method of claim 15 wherein the tube has an inner diameter at the interface between the heavy phase and the light phase of less than 1 μm.

22. The method of claim 15 wherein the tube is centrifuged at a rate of less than 10,000 G.

23. The method of claim 15 wherein after drawing the volume of the second fluid into the open-bottom tube, the portion of the tube proximal to the light phase is heat sealed across the portion containing the light phase to form a closed-bottom tube.

24. The method of claim 13 wherein the tube comprises a material selected from the group consisting of polyethylenes (PE), high-density polyethylenes (HDPE), low-density polyethylenes (LDPE), polyethylene terephthalates (PET), polypropylenes (PP), polyvinyl chlorides (PVC), polyvinylidene chlorides (PVDC), polystyrenes (PS), high impact polystyrene (HIPS), acrylonitrile butadiene styrenes, polyamides, polycarbonates, polyurethanes and nylons.

25. The method of claim 13 wherein at least 90% of the mitochondria in the concentrated mitochondrial preparation are functional.

26. A concentrated mitochondrial preparation comprising a solution including at least one mitochondrion per picoliter.

27. The concentrated mitochondrial preparation of claim 26, wherein the solution includes between one and 10,000 mitochondria per picoliter.

28. The concentrated mitochondrial preparation of claim 26 wherein the preparation further comprises a volume of a biocompatible oil.

29. The concentrated mitochondrial preparation of claim 26 wherein at least 90% of the mitochondria are functional.
